(19) United States
(12) Patent Application Publication
(10) Pub. No.: US 2007/0027522 A1
(43) Pub. Date: Feb. 1, 2007

(54) Stent delivery and guidewire systems

(76) Inventors: Jean C. Chang, Saratoga, CA (US); Sudip R. Pandya, Sunnyvale, CA (US)

Correspondence Address: BOZICEVIC, FIELD & FRANCIS LLP (CARDIOMIND), 1900 UNIVERSITY AVENUE, SUITE 200, EAST PALO ALTO, CA, 94303, US

(21) Appl. No.: 11/439,378

(22) Filed: May 22, 2006

Related U.S. Application Data
(63) Continuation of application No. 11/241,802, filed on Sep. 29, 2005.
(60) Provisional application No. 60/690,937, filed on Jun. 14, 2005.

(51) Int. Cl. A61F 2/06

(52) U.S. Cl. 623/1.11

(57) ABSTRACT

Medical device and methods for delivery or implantation of prostheses within hollow body organs and vessels or other luminal anatomy are disclosed. The subject technologies may be used in the treatment of atherosclerosis in stenting procedures.

### CROSS-REFERENCE TO RELATED APPLICATIONS

This application claims the benefit of U.S. Provisional Application Ser. No. 60/690,937, filed on Jun. 14, 2005, and U.S. patent application Ser. No. 11/241,802 filed on Sep. 29, 2005, incorporated by reference in their entireties.

### BACKGROUND OF THE INVENTION

Implants such as stents and occlusive coils have been used in patients for a wide variety of reasons. One of the most common “stenting” procedures is carried out in connection with the treatment of atherosclerosis, a disease that results in a narrowing and stenosis of body lumens, such as the coronary arteries. At the site of the narrowing (i.e., the site of a lesion) a balloon is typically dilatated in an angioplasty procedure to open the vessel. A stent is set in apposition to the interior surface of the lumen in order to help maintain an open passageway. This result may be affected by means of scaffolding support alone or in coordinated use with one or more drugs carried by the stent to aid in preventing restenosis.

Examples of self-expandable stents currently in use are the Magic WALLSTENT® and Radius™ stents (Boston Scientific/SCIMED Life Systems). Additional self-expanding stent background is presented in: “An Overview of Superelastic Stent Design,” Min. Invas Ther & Allied Technol 2002: 9(3/4) 235-246; “A Survey of Stent Designs,” Min. Invas Ther & Allied Technol 2002: 11(4) 137-147; “Coronary Artery Stents: Design and Biologic Considerations,” Cardiology Special Edition, 2003: 9(2) 9-14 and “Clinical and Angiographic Efficacy of a Self-Expanding Stent” Am Heart J 2003: 145(5) 868-874.

Because self-expanding prosthetic devices need not be set over a balloon (as with balloon-expandable designs), their delivery systems can be designed to a relatively smaller outer diameter than balloon-expandable counterparts. As such, self-expanding stents may be better suited to reach the smallest vasculature or achieve access in more difficult cases.

To realize such benefits, however, there continues to be a need in developing improved stents and stent delivery systems. Problems encountered with known delivery systems include drawbacks ranging from failure to provide means to enable precise stent placement, to bulkiness of system design. Inefficient design prohibits scaling systems to sizes as small as necessary to enable difficult access or small-vessel procedures (i.e., in tortuous vasculature or vessels having a diameter less than 3 mm, even less than 2 mm).

Sheath/pusher stent delivery systems are fairly space efficient. Examples are presented in U.S. Pat. No. 4,830,003 (Wolff, et al.) and U.S. Pat. No. 5,064,435 (Porter). In each, an outer sheath restraining a stent overrides an inner tubular member. The tubular member has a lumen adapted to receive a guidewire and a distal end adapted to abut the stent for delivery. A system capable of such use is also described in U.S. Pat. No. 4,580,568 (Gianturco) in which a sheath overrides a polymeric tubular member.

U.S. Pat. No. 6,280,465 (Cryer) discloses a very similar system. The device described in connection with FIG. 4 includes a central guidewire member, over which a tubular sheath and pusher are disposed. In use, the guidewire/pusher/sheath combination is advanced to a treatment site within a guiding catheter as an integral assembly. The ability to mount the stent and its retention means to any guidewire is expressed as desirable. Unit preassembly is also discussed as advantageous for time savings.

Irrespective of their various asserted advantages, all of these known sheath/pusher systems are limited in the degree to which they can be miniturized. The limiting factor is that the pusher must have sufficient wall thickness to offer an adequate interface to abut the stent when withdrawing the sheath or when pushing the stent out of the sheath.

U.S. Pat. No. 6,042,589 (Marianne) discloses a stent delivery system for employing a sheath/pusher type arrangement with the addition of an expandable balloon element for stabilizing the proximal end of the stent as the distal end of the stent opens concurrent with sheath withdrawal. The inclusion of the balloon further compounds the difficulty one would face in miniaturizing such a system.

Another system is disclosed in U.S. Pat. No. 6,989,024 (Hebert). Here, a stent is carried on a guidewire core member. A simple sheath is provided over the core member, covering the stent. Marker bands are optionally affixed to the core member, adjacent to the stent. The markers may serve to maintain stent position relative to the guidewire. Superelastic (SE) Nitinol is expressly contemplated for use in the guidewire body of the delivery system in the '156 publication, while shape memory alloy (SMA) Nitinol—alone—is disclosed for use in the stent. In the opinion of the assignee hereof, although the stent is often described as “self-expanding” in the subject publication, the written description of the application (including the text and drawings) only describes an SMA mode of self-expansion for the stent.

SMA Nitinol retains a deformed shape until the alloy is heated to thermodynamically drive a phase transition that restores the undeformed shape. In contrast, SE Nitinol can be flexed and will return immediately to shape upon release, springing back from strain of up to about 8%. Thus, when an SE Nitinol stent is being deployed by a sheath-based delivery system, stent expansion occurs progressively/concurrently with sheath removal. Numerous patents illustrate such activity—which activity differs from that disclosed in the '156 publication.

In the '156 publication, a situation is depicted in which the stent remains unchanged in configuration even after its sheath is removed (see, FIG. 6B). Then, with the stent exposed to warm blood flow in the vascular environment, heat exchange occurs thereby expanding the stent. Alternatively, the publication describes actuating expansion of the stent by application of electrical current after removal of the sheath.

A feature of self-expanding SE Nitinol stents is that they open to the greatest extent possible when confined in a restraining member such as a sheath. Stated otherwise, a SE stent forces/strains against its confining member.

Conversely, a stent employing SMA properties for self-expansion will remain in a collapsed state until heat-activated to drive it open. The '156 publication is believed to illustrate such a situation. The publication shows the stent set well inside its available envelope as defined by the inner wall of the sheath prior to stent delivery. That is, with stent delivery system in its pre-deployment configuration, a substantial gap exists between the outside of the stent and the inside of the sheath. Likewise, the marker/blocker features on the guidewire core member are set a substantial distance apart from the wall of the sheath. Because the marker/blocker features need only stabilize a fully-collapsed SMA stent (as opposed to an SE Nitinol stent straining to a maximal outer diameter) the blocker arrangement and stent/sheath gap illustrated are consistent with the other teachings of the '156 publication directed to SMA Nitinol stents.

As is commonly known, stents relying on shape memory alloy (SMA) thermally-driven shape recovery/change to open can be disadvantageous for reasons ranging from unpredictable deployment (due to even small variations in A_{finish} temperature, for reason of inadvertent heating during deployment, etc.) to a requirement that environmental controls be employed in device storage. Accordingly, there continues to be interest in developing space-efficient elastic or superelastic stent delivery systems. The present invention addresses this interest and offers other advantages as will be appreciated by one with skill in the art in view of the following disclosure.

### SUMMARY

In any medical procedure, saving surgical steps offers advantages both in terms of economic efficiency and improving patient care by requiring less time engaging in invasive activity. In stent procedures, over-the-wire stent delivery systems can offer such benefits. With a system that is able to be advanced over a guidewire and later removed following stent deployment, one avoids the need for exchanging the guidewire for the delivery device before and/or after the stent delivery procedure. The present invention offers such benefits, but in a higher performance package able to access and deliver one or more stents to sites including the neurovasculature, especially within the brain, and small vessels, particularly distal coronary arteries.

In accordance with the present invention, a delivery guide system is provided for use in delivering an implantable device to within the body. The subject systems are particularly useful for delivery and deploying a stent within the vasculature. The delivery guide system includes a corewire that can be used as a guidewire subsequent to implant delivery.

The corewire-turned-guidewire advantageously comprises a commercially available guidewire, a clone of such a wire or one offering comparable performance. As such, the member is tapered from a larger diameter at a more proximal end to a smaller diameter at a distal end up to an optional coil tip for use in tortuous or otherwise difficult to access anatomy. The “taper” may be a continuous taper or taper/step-down in size over sections. In certain embodiments, the corewire provides additional functions or carries components in addition to the stent. Specifically, the corewire may provide a filter device (e.g., an embolic filter) which is usable prior to (e.g., during an angioplasty procedure), during and/or after stent deployment. The corewire may further include radiopaque markers at selected locations along its distal length to demark, for example, the very distal tip, the filter location and/or the stent location.

The present invention provides a delivery system that may have a distal diameter of about 2 Fr (about 0.022 to 0.026 inch) or less and is adapted to deliver elastic/superelastic self expanding stents. The guidewire core member of the device preferably has a 0.014 to about 0.018 inch crossing profile. In this way, once the corewire is freed for use as a guidewire, it can be used with standard balloon catheter and microcatheter components.

An inner sleeve or tubular member is provided over the corewire/guidewire. An outer sleeve or tubular sheath is provided to restrain one or more stents carried by the delivery device. The inner sleeve serves to fill space between the guidewire core and external sheath. Employing an inner sleeve as opposed to a thicker wall sheath and/or an increased diameter core member offers a number of advantages ranging from system preparation to flexibility/trackability performance as elaborated upon below in connection with the drawings.

The inner sleeve may also serve in coordinated use with a raised feature on the corewire as a combination stent stop, blocker or abutment interface. An advantage of the combined sleeve/core feature is that it offers a relatively smaller diameter “bump” on the guidewire. A larger stop/blocker feature is required in instances where the inner sleeve stops short of the blocker feature, since that feature must—alone—offer a sufficient stop or abutment surface to stabilize the elastic or superelastic self-expanding stent for delivery. The raised stop feature may be a band connected (glued, welded, etc.) to the guidewire, a step or shoulder integral with the guidewire or be otherwise provided.

In any case, the raised stop feature comprises a solid body of unexpandible or at least substantially non-compressible material (e.g., it is not a balloon, gel or other compliant material) such as metal, plastic or a relatively high durometer electrometric material. It is a member designed to serve its function while occupying a minimal amount of space and/or have a minimal impact on the sizing of adjacent structure (e.g., it has no lumen leading thereto). Whether the raised feature has a scalloped shape, a perforate body or another physical form, it must offer a surface to abut and stabilize at least a portion of a proximal side of the stent. Generally, the raised feature will have a diameter between about 0.0015 and about 0.010 inches greater than that of an adjacent stent-side section of the corewire where the stent is received in the delivery system. In a system employing about a 0.014 inch guidewire core, the raised feature is generally about 0.0015 to about 0.0025 inch “tall”; in a system using about an 0.018 inch guidewire core, the raised feature is generally about 0.002 to about 0.005 inch tall; and in a system using a guidewire core of about 0.022 inch or larger, the raised feature is generally about 0.005 to about 0.010 inch tall.

After stent delivery by partial withdrawal of the outer sleeve, each of the inner and outer sleeves may be removed. With the device utilizing the combination blocker approach, the stent abutment feature then has a profile which is low enough so that it does not interfere with subsequent use of the core member as a fully functional guidewire. In this manner, a balloon catheter or another member can be advanced over the core member after removal of the other system components. Especially where the abutment/blocker member steps-up by about 0.002 inch over an adjacent section (i.e., when it is 0.004 greater in diameter for round sections), then a ramp is advantageously provided on the proximal side of the feature to provide an improved transition.

Optional medical procedure steps to be accomplished after stent delivery may include maintaining wire position while advancing another stent delivery system over the wire (specifically an over-the-wire delivery system different than the systems described herein), advancing a balloon catheter for a post-dilatation step, navigating to a new treatment site, etc.

In instances where the inner sleeve terminates proximal to the blocker member and/or the blocker member may be too large to allow a catheter to pass over the feature, the corewire still offers certain utility. For example, the wire may be advanced so that the blocker is distal to the stent delivered and next advancing a balloon catheter to effect post-dilatation at the lesion site.

While there may be circumstances in which the corewire should not or cannot be advanced as described, this variation of the invention may be desirable for reasons of ease of construction and robustness in design. Yet, the other variation of the invention facilitates a more complete set of options for using the guidewire after its sleeve elements are removed.

Irrespective of which one of the two designs are employed, to facilitate use of the corewire as a guidewire after stent delivery, it is contemplated that the tubular members are split or splitable. If the inner member is pre-split or includes perforations or other features to aid in splitting open the sleeve, the open or perforated, etc. length will generally terminate within about 5 to about 15 cm of a distal end for the sake of stability or component strength at the end of the device that interfaces with the stent. The same may be true of the distal section of the outer tubular member. With a pre-split inner member, (either fully or partially), the system relies on the outer member to hold the components together. In another example, the handle used in the system includes a blade or wedge member to cut or assist in opening the sleeves as they are pulled through the handle.

If not fully split or splittabe such that they can simply be pulled off the corewire directly, the sleeves may instead be withdrawn proximally up to a point where any closed portion remains over the guidewire/corewire. A physician may then switch his grip from a proximal location to the sleeve portions, to distal of them—even with a wire between about 150 and about 180 cm in length. Alternatively, a longer 300 cm “exchange length” wire or “dock” type system could be used to provide an overall length that allows the sleeves to be withdrawn clear of the wire while holding a proximal portion of the device/assembly. In any case, the option of removing the inner and outer sleeves from the corewire of the device offers a physician a bare wire (upon optional handle removal) for use in a vessel without altering or disturbing a distal position of the wire.

Yet, the wire starts out as an integral part of the delivery system. As such, it is specifically sized for optimal use with the other components and includes those blocker/stop features noted above. Such construction lends itself to providing a system that is torquable en masse and/or one in which the corewire can be set to be spun/rotated within the sleeves to specifically direct the tip.

The present invention includes systems comprising any combination of the features described herein. Methodology described in association with the devices disclosed also forms part of the invention. Such methodology may include that associated with completing an angioplasty, bridging an aneurysm, deploying radially-expandable anchors for pacing leads or an embolic filter, or placement of a prosthesis within neurovasculature, an organ selected from the kidney and liver, within reproductive anatomy such as selected vas deferens and fallopian tubes or other applications.

### Definitions

The term “stent” as used herein refers to any coronary artery stent, other vascular prosthesis, or other radially expanding or expandable prosthesis or scaffold-type implant suitable for the noted treatments or otherwise. Exemplary structures include wire mesh or lattice patterns and coils, though others may be employed in the present invention.

A “self-expanding” stent as used herein is a scaffold-type structure (serving any of a number of purposes) that expands from a reduced-diameter (be it circular or otherwise) configuration to an increased-diameter configuration by elastic or pseudoelastic recovery in response to removal of a restraining member. Accordingly, when held by the restraint, the stent strains or presses against the inner wall of the restraint structure. As such, neither the alloy nor the delivery system is configured so that the stent will retain its shape within the body without restraint. In other words, where an alloy such as Nitinol is used in a stent according to the present invention, its A_{finish} temperature is at body temperature or below (i.e., less than or equal to about 37 degrees C.)

A “wire” as used herein generally comprises a common metallic member. However, the wire may be coated or covered by a polymeric material (e.g., with a lubricious material such as TEFLON®, i.e., PTFE or PolyTetraFlouroEthelyne) or otherwise. Still further, the “wire” may be a hybrid structure with metal and a polymeric material (e.g., Vectra™, Spectra™, Nylon, etc.) or composite material (e.g., carbon fiber in a polymer matrix). The wire may be a filament, bundle of filaments, cable, ribbon or in some other form. It is generally not hollow.

A “guidewire” or “corewire” as used herein generally comprises member tapered or stepping down from an enlarged proximal diameter to a reduced distal diameter. It generally terminates in an atraumatic tip that may have a diameter equal to or greater than a proximal section of the wire. The dimensions and relative length and location of the two or more different diameter sections, tapers between them, as well as the parameters (length, angle, etc.) may vary. Likewise, material selection may vary. In one example a 0.014 inch wire has a proximal shaft of about a 0.014 inch diameter, a reduced diameter distal section of about a 0.010 inch diameter and a coil tip having about a 0.014 inch diameter.

A “hypotube” or “hypotubing” as referred to herein refers to hypodermic needle tubing or other small diameter tubing in the size range discussed below, generally with a thin wall. The hypotube may specifically be hypodermic needle tubing. Alternatively, it may be wound or braided cable tubing, such as provided by Asahi Intec Co., Ltd or otherwise. As with the “wire” discussed above, the material defining the hypotube may be metallic, polymeric or a hybrid of metallic and polymeric or composite material.

An “atraumatic tip” may comprise a plurality of spring coils attached to a tapered wire section. At a distal end, the coils typically terminate with a bulb or ball that is often made of solder. In such a construction, the coils and/or solder is/are often platinum alloy or another radiopaque material. The coils may also be platinum, or be of another material. In the present invention, the wire section to which the coils are attached may be tapered, but need not be tapered. In addition, alternate structures are possible. For instance, molding or dip-coating with a polymer may be employed. In one example, the atraumatic tip may comprise a molded tantalum-loaded 35 durometer Pebax™ tip. However constructed, the atraumatic tip may be straight or curved, the latter configuration possibly assisting in directing or steering the delivery guide to a desired intravascular location.

“Radiopaque markers” are understood to be markers or features of the various delivery system components, corewire or implant that may be employed to facilitate visualization of the system components. As such, various platinum (or other radiopaque material) bands, coatings or other markers (such as tantalum plugs) may be variously incorporated into the system. Alternatively, or additionally, the stent may be made of radiopaque material or incorporate them. Especially where the stent employed may shorten somewhat upon deployment, it may also be desired to align radiopaque features with the expected location (relative to the body of the inner member) of the stent upon deployment. A filter used with the subject devices may also be made of radiopaque material for the same reasons.

To “attach”, “connect” or to have or make a “connection” or “attachment” between parts refers to fusing, bonding, welding (by resistance, laser, chemically, ultrasonically, etc), gluing, pinning, crimping, clamping or otherwise mechanically or physically joining, attaching or holding components together (permanently or temporarily).

All existing subject matter mentioned herein (e.g., publications, patents, patent applications and hardware) is incorporated by reference herein in its entirety to provide additional context to the present invention except insofar as the subject matter may conflict with that of the present invention (in which case what is present herein shall prevail). Note that the referenced items are provided solely for their disclosure prior to the filing date of the present application. Nothing herein is to be construed as an admission that the present invention is not entitled to antedate such material by virtue of prior invention.

### BRIEF DESCRIPTION OF THE DRAWINGS

Each of the figures diagrammatically illustrates aspects of the invention. Of these:

FIG. 1 shows a heart in which its vessels may be the subject of one or more angioplasty and stenting procedures;

FIG. 2A shows an expanded stent cut pattern as may be used in producing a stent according to a first aspect of the invention; FIG. 2B shows a stent cut pattern for a second stent produced according to another aspect of the present invention;

FIG. 3A shows an expanded stent cut pattern as may be used in producing a stent according to a first aspect of the invention; FIG. 3B shows a stent cut pattern for a second stent produced according to another aspect of the present invention;

FIGS. 4A-4L illustrate stent deployment methodology to be carried out with the subject delivery guide member; alternative stent deployment acts are shown in FIGS. 4D′-4I′.

FIGS. 5A and 5B show distal sectional views of delivery systems according to the present invention, together with detail views as indicated;

FIG. 6 shows a handle as may be used in the present invention;

FIG. 7 shows the handle of FIG. 6 in cross-section, together with detail views as indicated; and

FIG. 8 shows another exemplary variation of a subject delivery system having a corewire provided with an embolic filter.

In the figures, like elements in some cases are indicated by a related numbering scheme. Furthermore, variation of the invention from the embodiments pictured is, of course, contemplated.

### DETAILED DESCRIPTION OF THE INVENTION

Various exemplary embodiments of the invention are described below. Reference is made to these examples in a non-limiting sense. They are provided to illustrate more broadly applicable aspects of the present invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. All such modifications are intended to be within the scope of the claims made herein.

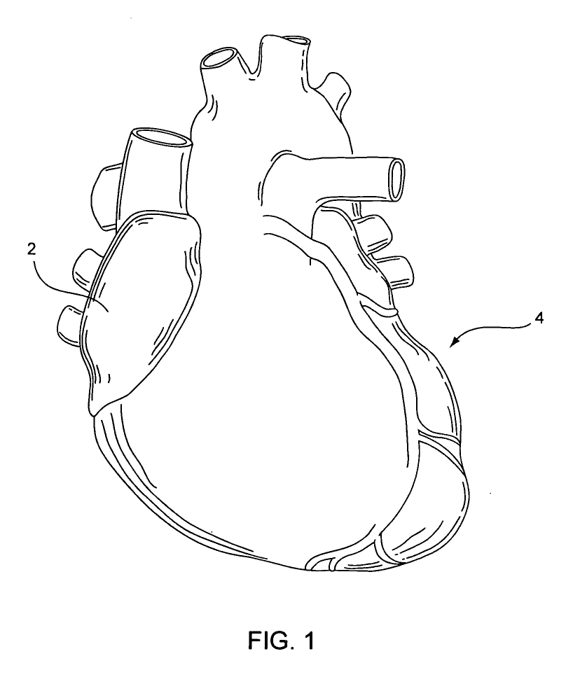

In light of this framework, FIG. 1 shows a heart 2 in which its vessels may be the subject of one or more angioplasty and/or stenting procedures. To date, however, significant difficulty or impossibility is confronted in reaching smaller coronary arteries 4. If a stent and a delivery system could be provided for accessing such small vessels and other difficult anatomy, an additional 20 to 25% of coronary percutaneous procedures could be performed with such a system. Such potential offers opportunity for huge gains in human healthcare and a concomitant market opportunity in the realm of roughly $1 billion U.S. dollars—with the further benefit of avoiding loss of income and productivity of those treated.

Features of the present invention are uniquely suited for a system able to reach small vessels (though use of the subject systems s not limited to such a setting.) By “small” coronary vessels, it is meant vessels having an inside diameter between about 1.5 or 2 and about 3 mm in diameter. These vessels include, but are not limited to, the Posterior Descending Artery (PDA), Obtuse Marginal (OM) and small diagonals. Conditions such as diffuse stenosis and diabetes produce conditions that represent other access and delivery challenges which can be addressed with a delivery system according to the present invention. Other extended treatment areas addressable with the subject systems include vessel bifurcations, chronic total occlusions (CTOs), and prevention procedures (such as in stenting of vulnerable plaque).

Assuming a means of delivering one or more appropriately-sized stents, it may be preferred to use a drug eluting stent (DES) in such an application to aid in preventing restenosis. A review of suitable drug coatings and available vendors is presented in “DES Overview: Agents, release mechanism, and stent platform” a presentation by Campbell Rogers, MD incorporated by reference in its entirety. However, bare-metal stents may be employed in the present invention.

While some might argue that the particular role and optimal usage of self expanding stents has yet to be defined, they offer an inherent advantage over balloon expandable stents. The latter type of devices produce “skid mark” trauma (at least when delivered uncovered upon a balloon) and are associated with a higher risk of end dissection or barotraumas caused at least in part by high balloon pressures and related forces when deforming a balloon-expandable stent for deployment.

Yet, with an appropriate deployment system, self-expanding stents may offer one or more of the following advantages over balloon-expandable models: 1) greater accessibility to distal, tortuous and small vessel anatomy—by virtue of decreasing crossing diameter and increasing compliance relative to a system requiring a deployment balloon, 2) sequentially controlled or “gentle” device deployment, 3) use with low pressure balloon pre-dilatation (if desirable) to reduce barotraumas, 4) strut thickness reduction in some cases reducing the amount of “foreign body” material in a vessel or other body conduit, 5) opportunity to treat neurovasculature—due to smaller crossing diameters and/or gentle delivery options, 6) the ability to easily scale-up a successful treatment system to treat larger vessels or vice versa, 7) a decrease in system complexity, offering potential advantages both in terms of reliability and system cost, 8) reducing intimal hyperplasia, and 9) conforming to tapering anatomy—without imparting complimentary geometry to the stent (though this option exists as well).

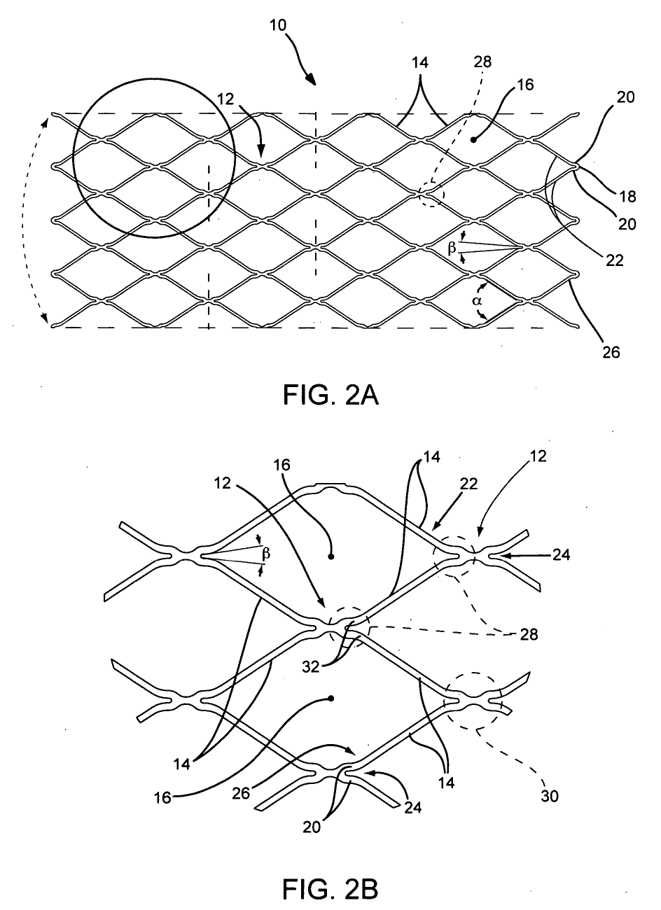

At least some of these noted advantages may be realized using a stent 10 as shown in FIG. 2A. The stent pattern pictured is well suited for use in small vessels. It may be collapsed to an outer diameter of about 0.018 inch (0.46 mm), or even smaller to about 0.014 inch (0.36 mm)—including the restraint/joint used to hold it down—and expand to a size (fully unrestrained) between about 1.5 mm (0.059 inch) or 2 mm (0.079 inch) or 3 mm (0.12 inch) and about 3.5 mm (0.14 inch).

In use, the stent will be sized so that it is not fully expanded when fully deployed against the wall of a vessel in order to provide a measure of radial force thereto (i.e., the stent will be “oversized” as discussed above). The force will secure the stent and offer potential benefits in reducing intimal hyperplasia and vessel collapse or even pinning dissected tissue in apposition.

Stent 10 preferably comprises NiTi that is superelastic at or below room temperature and above (i.e., as in having an A_{f} as low as 15 degrees C. or even 0 degrees C.). Also, the stent is preferably electropolished. The stent may be a DES unit. The drug can be directly applied to the stent surface(s), or introduced into pockets or an appropriate matrix set over at least an outer portion of the stent. The stent may be coated with gold and/or platinum to provide improved radiopacity for viewing under medical imaging.

For a stent able to collapse to an outer diameter of about 0.012 inches and expand to about 3.5 mm, the thickness of the NiTi is about 0.0025 inch (0.64 mm). Such a stent is designed for use in a 3 mm vessel or other body conduit, thereby providing the desired radial force in the manner noted above. Further information regarding radial force parameters in coronary stents may be noted in the article, “Radial Force of Coronary Stents: A Comparative Analysis,” Catheterization and Cardiovascular Interventions 46: 380-391 (1999), incorporated by reference herein in its entirety.

In one manner of production, the stent in FIG. 2A is laser or EDM cut from round NiTi tubing, with the flattened-out pattern shown wrapping around the tube as indicated by dashed lines. In such a procedure, the stent is preferably cut in its fully-expanded shape. By initially producing the stent to full size, the approach allows cutting finer details in comparison to simply cutting a smaller tube with slits and then heat-expanding/annealing it into its final (working) diameter. Avoiding post-cutting heat forming also reduces production cost as well as the above-reference effects.

Regarding the finer details of the subject stent, as readily observed in the detail view provided in FIG. 2B, necked down bridge sections 12 are provided between axially/horizontally adjacent struts or arms/legs 14, wherein the struts define a lattice of closed cells 16. Terminal ends 18 of the cells are preferably rounded-off so as to be atraumatic.

To increase stent conformability to tortuous anatomy, the bridge sections can be strategically separated or opened as indicated by the broken lines in FIG. 2A. To facilitate such tuning of the stent, the bridge sections are sufficiently long so that fully rounded ends 18 may be formed internally to the lattice just as shown on the outside of the stent if the connection(s) is/are severed to separate adjacent cells 16. Whether provided as ends 18 or adjoined by a bridge section 12, junction sections 28 connect circumferentially or vertically adjacent struts (as illustrated). Where no bridge sections are provided, the junction sections can be unified between horizontally adjacent stent struts as indicated in region 30.

The advantage of the optional double-concave profile of each strut bridge 12 is that it reduces material width (relative to what would otherwise be presented by a parallel side profile) to improve flexibility and thus trackability and conformability of the stent within the subject anatomy while still maintaining the option for separating/breaking the cells apart.

Further optional features of stent 10 are employed in the cell end regions 18 of the design. Specifically, strut ends 20 increase in width relative to medial strut portions 22. Such a configuration distributes bending (during collapse of the stent) preferentially toward the mid region of the struts. For a given stent diameter and deflection, longer struts allow for lower stresses within the stent (and, hence, a possibility of higher compression ratios). Shorter struts allow for greater radial force (and concomitant resistance to a radially applied load) upon deployment.

In order to increase stent compliance so that it collapses as much as possible, accommodation is made for the stiffer strut ends 20 provided in the design shown in FIG. 2A. Namely, the gap 24 between the strut ends 22 is set at a smaller angle as if the stent were already partially collapsed in that area. Thus, the smaller amount of angular deflection that occurs at ends 20 can bring the sections parallel (or nearly so) when the strut medial portions 22 are so-arranged. In the variation of the invention in FIG. 2A, radiused or curved sections 26 provide a transition from a medial strut angle α (ranging from about 85 degrees to about 60 degrees) to an end strut angle β (ranging from about 30 to about 0 degrees) at the strut junctions 28 and/or extensions therefrom.

In addition, it is noted that gap 24 and angle β may actually be configured to completely close prior to fully collapsing angle α. The stent shown is not so-configured. Still, the value of doing so would be to limit the strains (and hence, stresses) at the strut ends 22 and cell end regions 18 by providing a physical stop to prevent further strain.

In the detail view of FIG. 2B, angle β is set at 0 degrees. The gap 24 defined thereby by virtue of the noticeably thicker end sections 20 at the junction result in very little flexure along those lever arms. The strut medial portions are especially intended to accommodate bending. In addition, a hinging effect at the corner or turn 32 of junction section 28 causing rotation of the struts largely about angle α may provide for compression mode in this stent.

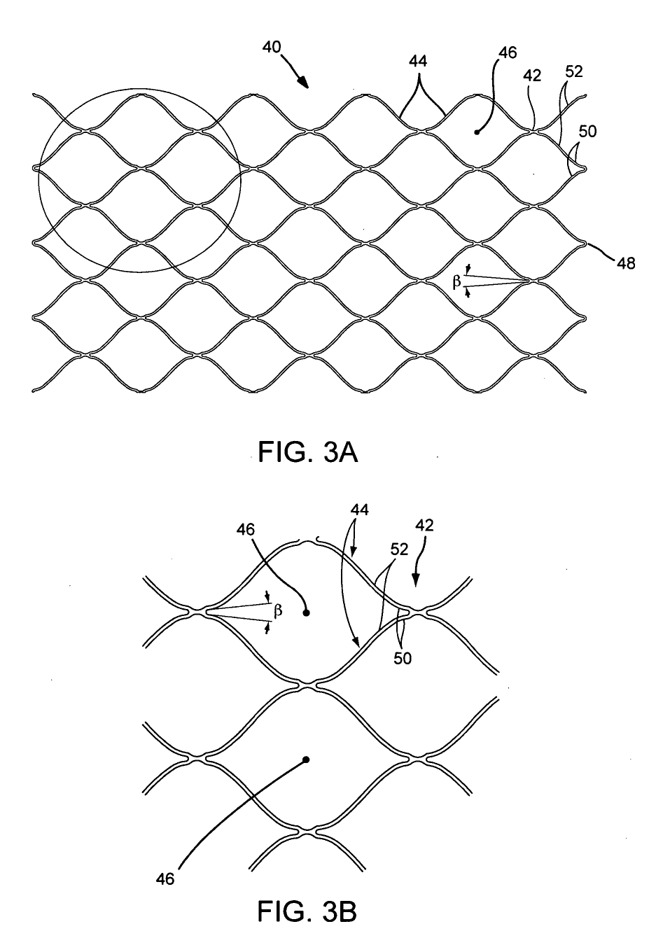

The stent pattern shown in FIG. 3A and detailed in FIG. 3B offers certain similarities as well as some major differences from the stent pattern presented in FIGS. 2A and 2B. As in the variation above, stent 40 includes necked down bridge sections 42 provided between adjacent struts or arms/legs 44, wherein the struts define a lattice of closed cells 46. In addition, terminal ends 48 of the cells are preferably rounded-off so as to be atraumatic.

Furthermore, the bridge sections 42 of stent 40 can be separated for compliance purposes. In addition, they may be otherwise modified (e.g., as described above) or even eliminated. Also, in each design, the overall dimensions of the cells and indeed the number of cells provided to define axial length and/or diameter may be varied (as indicated by the vertical and horizontal section lines in FIG. 3A).

Like the previous stent design, strut ends 50 may offer some increase in width relative to medial strut portions 52. However, as shown in FIG. 3B, as compared to FIG. 2B, the angle β is relatively larger. Such a configuration is not concerned with developing a hinge section and a relatively stiffer outer strut section. Instead, angle β in the FIG. 3A/3B design is meant to collapse and the strut ends are meant to bend in concert with the medial strut portions so as to essentially straighten-out upon collapsing the stent, generally forming tear-drop spaces between adjacent struts. This approach offers a stress-reducing radius of curvature at strut junctures as well as maximum stent compression.

The “S” curves defined by the struts are produced in a stent cut to a final or near final size (as shown in FIGS. 3A and 3B). The curves are preferably determined by virtue of their origination in a physical or computer model that is expanded from a desired compressed shape to the final expanded shape. So derived, the stent can be compressed or collapsed under force to provide an outer surface profile that is as solid or smooth and/or cylindrical as possible or feasible.

Such action is enabled by distribution of the stresses associated with compression to generate stains to produce the intended compressed and expanded shapes. This effect is accomplished in a design unaffected by one or more expansion and heat setting cycles that otherwise deteriorate the quality of the superelastic NiTi stent material. Further details regarding the “S” stent design and alternative stent constructions as may be used in the present invention are disclosed in U.S. Provisional Patent Application Ser. No. 60/619,437, entitled, “Small Vessel Stent Designs”, filed Oct. 14, 2004 and incorporated herein by reference in its entirety. In the case of each of the above stent designs, by utilizing a stent design that minimizes problematic strain (and in the latter case actually uses the same to provide an improved compressed profile), very high compression ratios of the stent may be achieved from about 5× to about 10× or above.

Delivery systems according to the present invention are advantageously sized to correspond to existing guidewire sizes. For example, the system may have about an 0.014 (0.36 mm), 0.018 (0.46 mm), 0.022 (0.56 mm), 0.025 (0.64 mm) inch crossing profile. Of course, intermediate sizes may be employed as well, especially for full-custom systems. Still further, it is contemplated that the system sizing may be set to correspond to French (FR) sizing. In that case, system sizes contemplated range at least from about 1 to about 2 FR, whereas the smallest known balloon-expandable stent delivery systems are in the size range of about 3 to about 4 FR.

In instances where the overall device crossing profile matches a known guidewire size, they may be used with off-the-shelf components such as balloon and microcatheters. As referenced above, the corewire member of the device is likewise advantageously so-sized for similar reasons as elaborated upon herein and other.

At least when produced in the smallest sizes (whether in an even/standard guidewire or FR size, or otherwise), the system enables a substantially new mode of stent deployment in which delivery is achieved through an angioplasty balloon catheter or small microcatheter lumen. Further discussion and details of “through the lumen” delivery is presented in U.S. patent application Ser. No. 10/746,455 “Balloon Catheter Lumen Based Stent Delivery Systems” filed on Dec. 24, 2003 and its PCT counterpart US2004/008909 filed on Mar. 23, 2004, each incorporated by reference in its entirety.

In larger sizes, (i.e., up to about 0.035 inch crossing profile or more), the system is most applicable to peripheral vessel applications as elaborated upon below. Yet, even in “small vessel” cases or applications (where the vessel to be treated has a diameter up to about 3.0 mm), it may also be advantageous to employ a stent delivery system sized at between about 0.022 to about 0.025 inch in diameter. Such a system can be used with catheters compatible with 0.022 and/or 0.025 inch diameter guidewires.

While such a system may not be suitable for reaching the very smallest vessels, this variation of the invention is quite advantageous in comparison to known systems in reaching the larger of the small vessels (i.e., those having a diameter of about 2.5 mm or larger). By way of comparison, among the smallest known over-the-guidewire delivery systems are the Micro-Driver™ by Medtronic and the Pixel™ systems by Guidant. These are adapted to treat vessels between 2 and 2.75 mm, the latter system having a crossing profile of 0.036 inches (0.91 mm). A system described in U.S. Patent Publication No. 2002/0147491 for treating small vessels is supposedly capable of downsizing to 0.026 inch (0.66 mm) in diameter. Furthermore, because the core member of the subject device can be used as a guidewire (in one fashion or another) after stent delivery, the present invention offers further advantages in use as elaborated upon below.

As referenced above, it may be desired to design a variation of the subject system for use in deploying stents in larger, peripheral vessels, biliary ducts or other hollow body organs. Such applications involve a stent being emplaced in a region having a diameter from about 3.5 to about 13 mm (0.5 inch). In which case, a 0.035 to 0.039 inch (3 FR) diameter crossing profile system is advantageously provided in which the stent expands (unconstrained) to a size between about roughly 0.5 mm and about 1.0 mm greater than the vessel or hollow body organ to be treated. Sufficient stent expansion is easily achieved with the exemplary stent patterns shown in FIGS. 2A/2B or 3A/3B.

Again, as a matter of comparison, the smallest delivery systems known to applicants for stent delivery in treating such larger-diameter vessels or biliary ducts is a 6 FR system (nominal 0.084 inch outer diameter), which is suited for use in an 8 FR guiding catheter. Thus, even in the larger sizes, the present invention affords opportunities not heretofore possible in achieving delivery systems in the size range of a commonly used guidewire, with the concomitant advantages discussed herein.

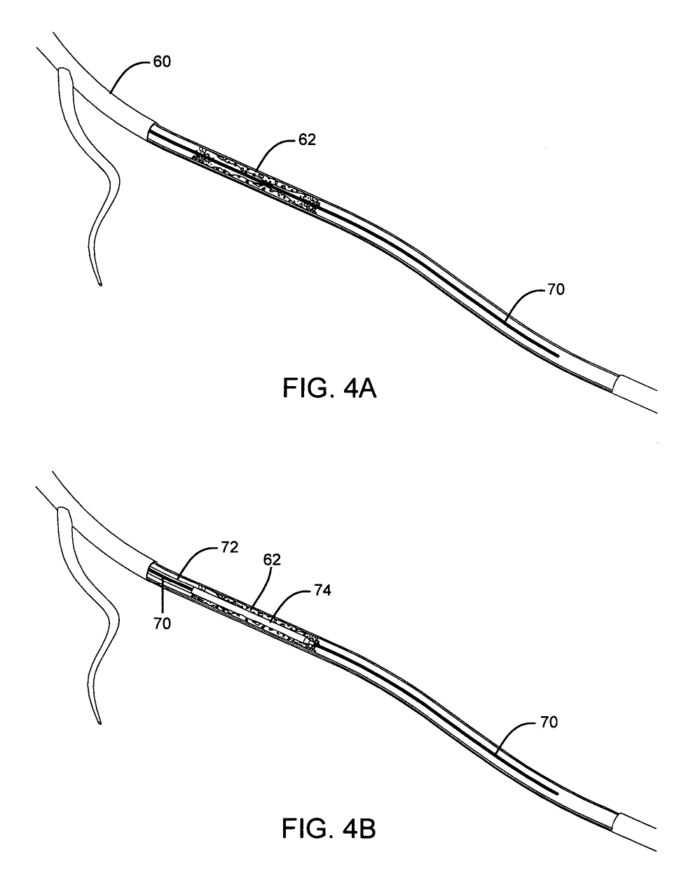

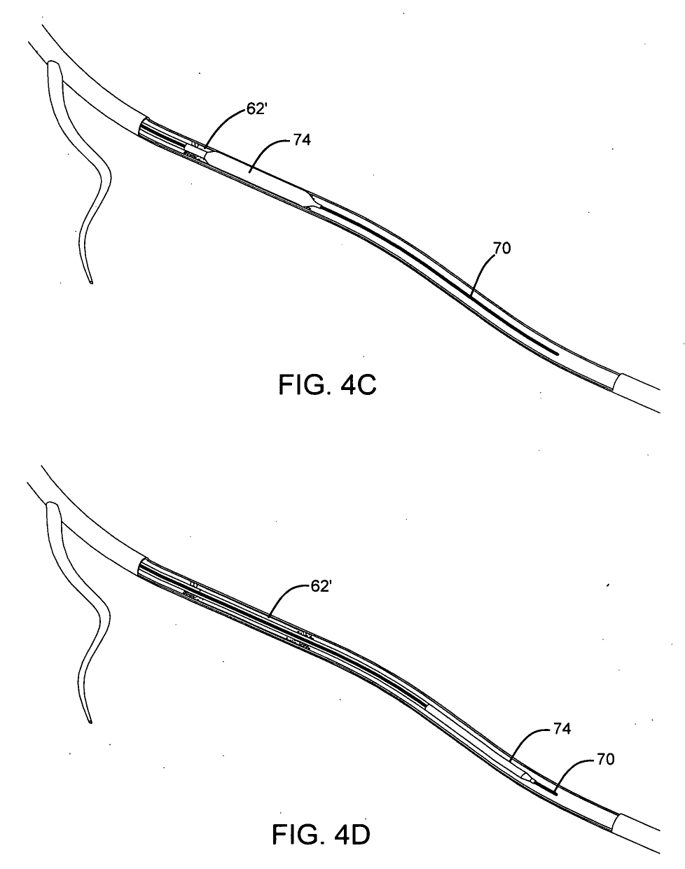

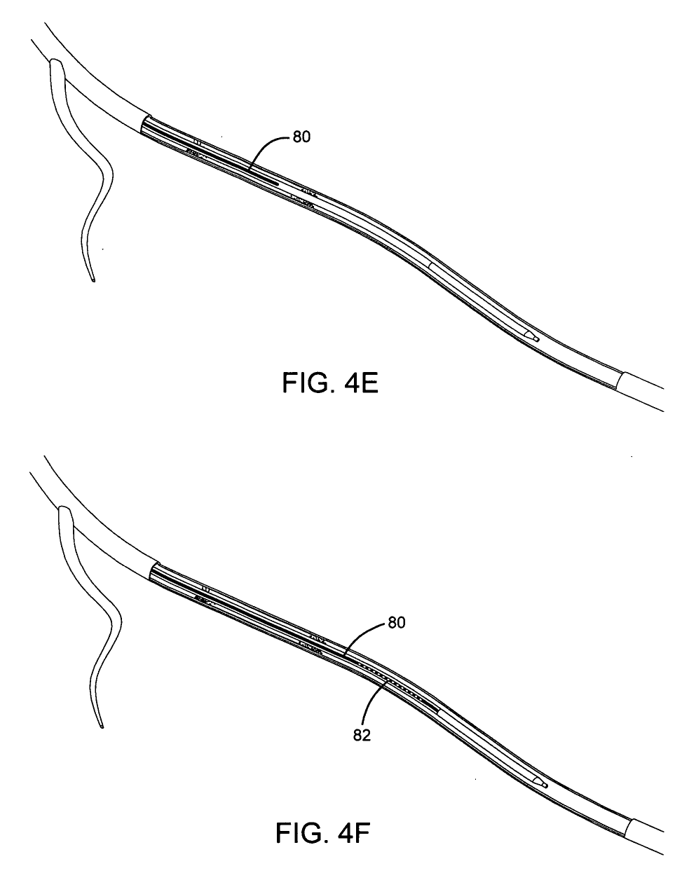

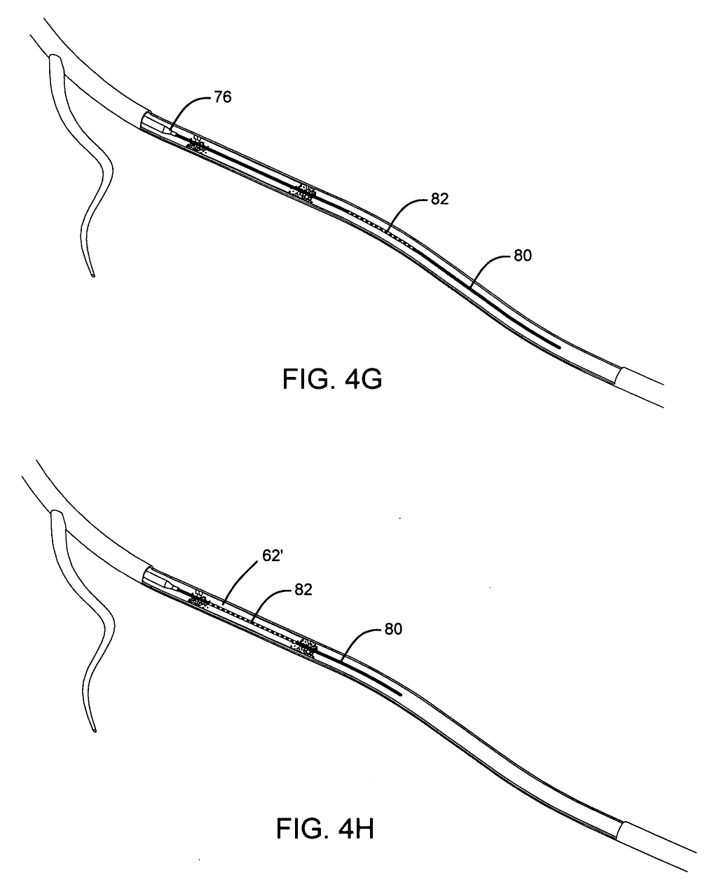

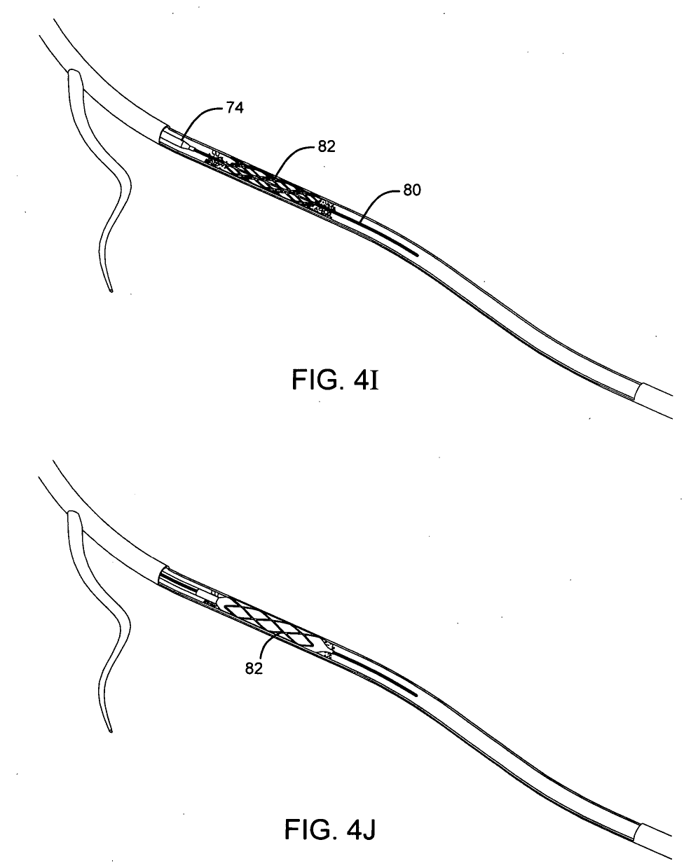

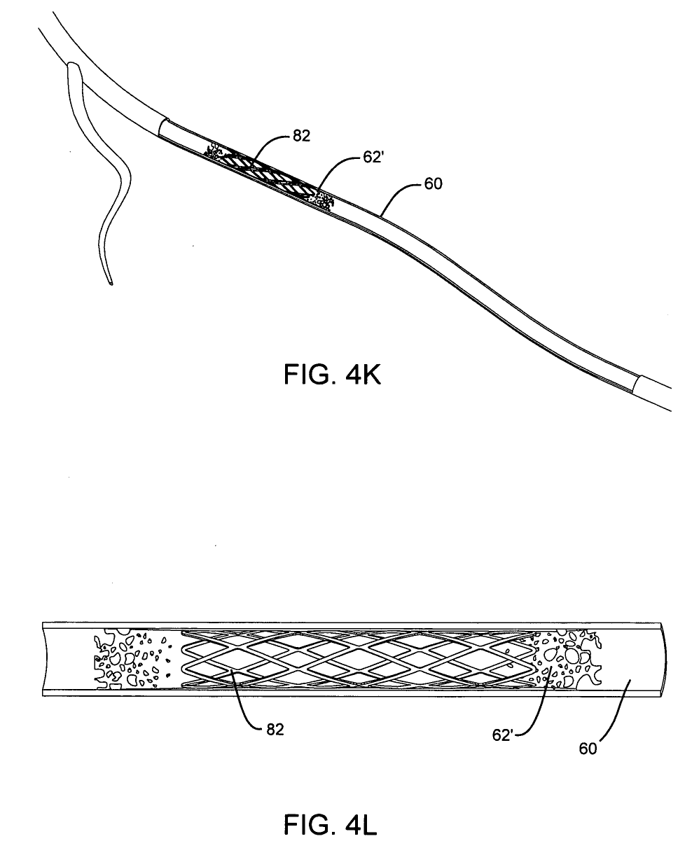

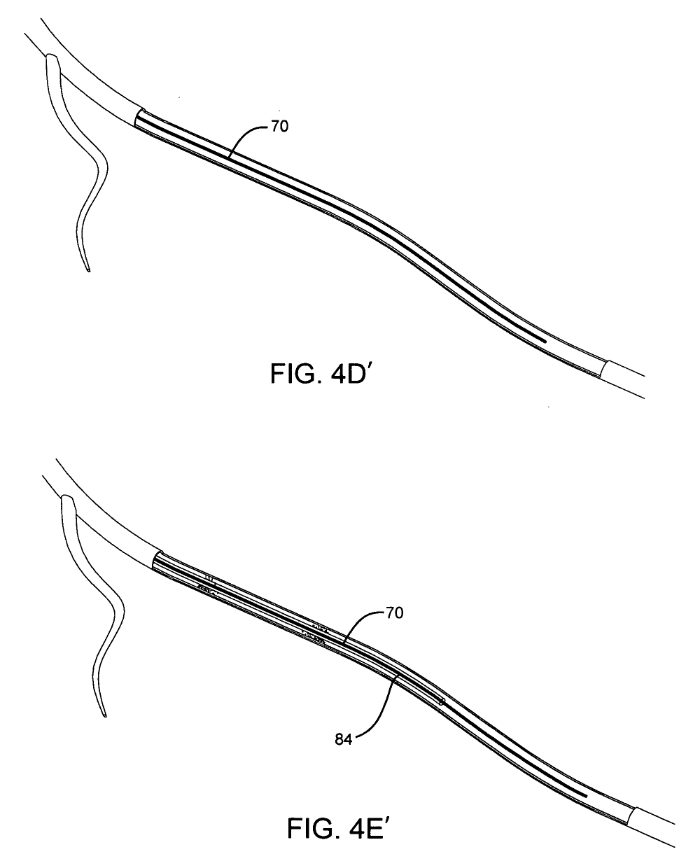

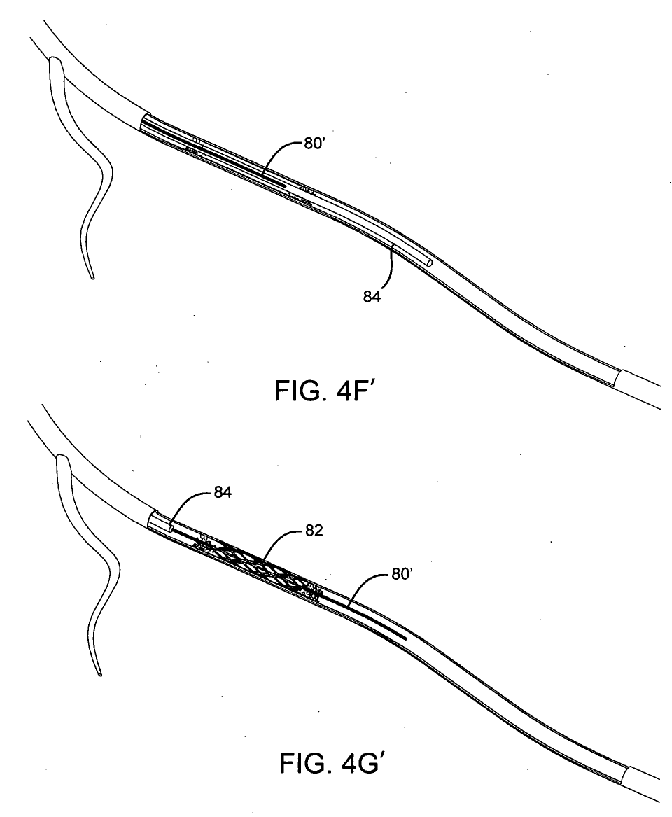

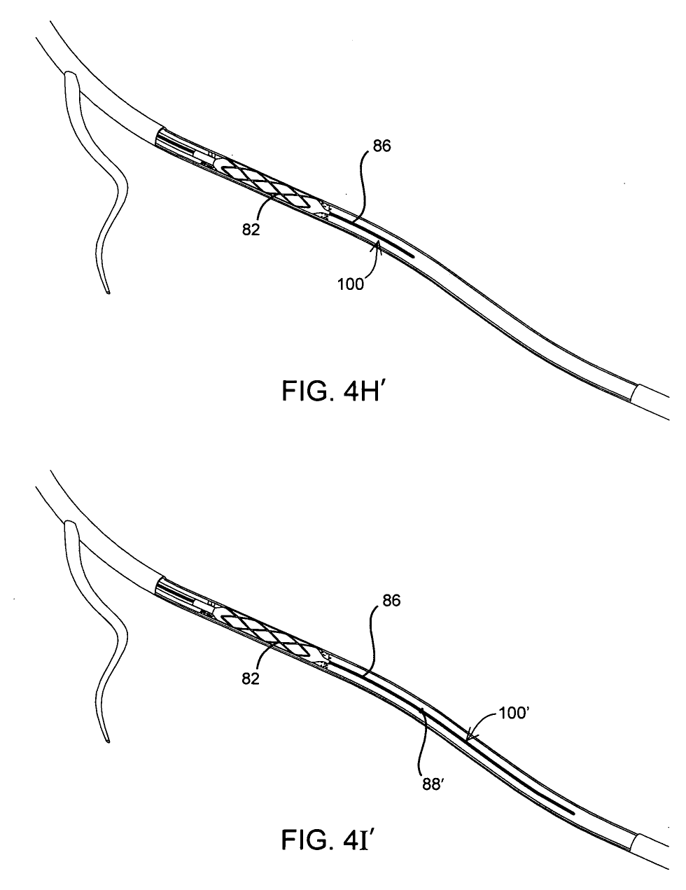

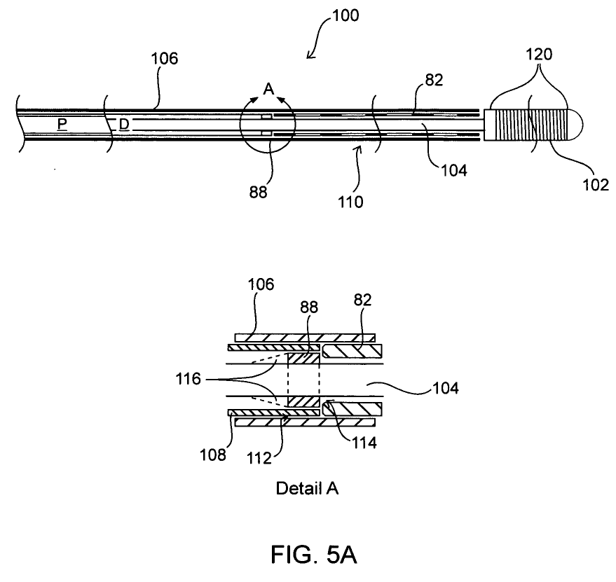

As for the manner of using the inventive system as optionally configured, FIGS. 4A-4L illustrate an exemplary angioplasty procedure. Still, the delivery systems and stents or implants described herein may be used otherwise—especially as specifically referenced herein.

Turning to FIG. 4A, it shows a coronary artery 60 that is partially or totally occluded by plaque at a treatment site/lesion 62. Into this vessel, a guidewire 70 is passed distal to the treatment site. In FIG. 4B, a balloon catheter 72 with a balloon tip 74 is passed over the guidewire, aligning the balloon portion with the lesion (the balloon catheter shaft proximal to the balloon is shown in cross section with guidewire 70 therein).

As illustrated in FIG. 4C, balloon 74 is expanded (dilatated or dialated) in performing an angioplasty procedure, opening the vessel in the region of lesion 62. The balloon expansion may be regarded as “predilatation” in the sense that it will be followed by stent placement (and optionally) a “postdilataton” balloon expansion procedure.

Next, for systems compatible (i.e., systems able to pass through a balloon catheter lumen) the balloon is at least partially deflated and passed forward, beyond the dilate segment 62′ as shown in FIG. 4D. At this point, guidewire 70 is removed as illustrated in FIG. 4E. It is exchanged for a delivery guide member 80 carrying stent 82 as further described below. This exchange is illustrated in FIGS. 4E and 4F.

However, it should be appreciated that such an exchange need not occur. Rather, the original guidewire device inside the balloon catheter (or any other catheter used) may be that of item 80, instead of the standard guidewire 70 shown in FIG. 4A. Thus, the steps depicted in FIGS. 4E and 4F (hence, the figures also) may be omitted.

In addition, there may be no use in performing the step in FIG. 4D of advancing the balloon catheter past the lesion, since such placement is merely for the purpose of avoiding disturbing the site of the lesion by moving a guidewire past the same. FIG. 4G illustrates the next act in either case. Particularly, the balloon catheter is withdrawn so that its distal end 76 clears the lesion. Preferably, delivery guide 80 is held stationary, in a stable position. After the balloon is pulled back, so is delivery device 80, positioning stent 82 where desired. Note, however, that simultaneous retraction may be undertaken, combining the acts depicted in FIGS. 4G and 4H. Whatever the case, it should also be appreciated that the coordinated movement will typically be achieved by virtue of skilled manipulation by a doctor viewing one or more radiopaque features associated with the stent or delivery system under medical imaging.

Once placement of the stent across from dilated segment 62′ is accomplished, stent deployment commences. The manner of deployment is elaborated upon below. Upon deployment, stent 82 assumes an at least partially expanded shape in apposition to the compressed plaque as shown in FIG. 4I. Next, the aforementioned postdilatation may be effected as shown in FIG. 4J by positioning balloon 74 within stent 82 and expanding both. This procedure may further expand the stent, pushing it into adjacent plaque—helping to secure each.

Naturally, the balloon need not be reintroduced for postdilatation, but it may be preferred. Regardless, once the delivery device 80 and balloon catheter 72 are withdrawn as in FIG. 4K, the angioplasty and stenting procedure at the lesion in vessel 60 is complete. FIG. 4L shows a detailed view of the emplaced stent and the desired resultant product in the form of a supported, open vessel.

In an alternative procedure approach, delivery system 80 is too large to pass through the lumen of a balloon catheter. In which case, the procedure follows another path. Specifically, instead of advancing the balloon catheter after dilatation as in FIG. 4D, it is instead withdrawn as shown in FIG. 4D′ lumen, the balloon catheter is withdrawn. Next, as shown in FIG. 4E′, a standard catheter/microcatheter 84 is advanced over original guidewire 70. Then, as shown in FIG. 4F′, the guidewire is exchanged for the delivery system 80′. With the delivery system in place and delivery catheter 82 withdrawn proximal of the lesion, the stent is deployed as shown in FIG. 4G′.

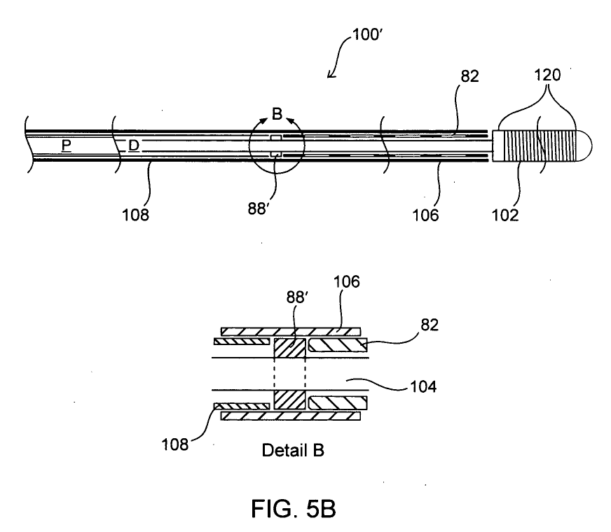

To enable subsequent steps, the delivery system may then be stripped down to its corewire 86 as elaborated upon below. Now with the remaining small-size wire, with a delivery system as described illustrated in connection with FIG. 5A below it is possible to exchange the microcatheter for a balloon catheter to effect post dilatation as shown in FIG. 4H′. Here, the balloon catheter 74 overrides the stent carrying region delivery device. When a delivery device 80′ is employed as described in connection with FIG. 5B, as shown in FIG. 4I′ the corewire 86 is advanced to a position so that stop feature 88 provided to block proximal motion of the stent upon sheath retraction will not interfere with advancing the balloon catheter to effect post post-dilatation.

It is also to be recognized that once it is freed from the sleeve portions of the delivery device, the corewire may be used for other subsequent procedures such as navigation to another target location for stenting, etc. In this way, the element functions as or substantially like a typical guidewire.

Furthermore, it is to be recognized that the subject invention may be practiced to perform “direct stenting.” That is, a stent may be delivered alone to maintain a body conduit, without preceding balloon angioplasty. Likewise, once one or more stents are delivered with the subject system (either by a single system, or by using multiple systems) the post-dilatation procedure(s) discussed above are merely optional. In addition, other endpoints may be desired such as implanting an anchoring stent in a hollow tubular body organ, closing off an aneurysm, delivering a plurality of stents, etc. In performing any of a variety of these or other procedures, suitable modification will be made in the subject methodology. The procedure shown is depicted merely because it illustrates a preferred mode of practicing the subject invention, despite its potential for broader applicability.

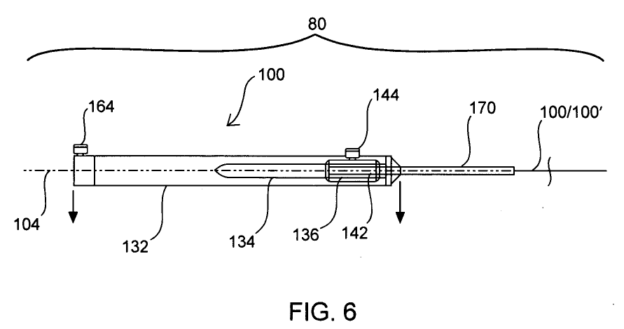

A more detailed understanding the subject delivery system is provided in FIGS. 5A and 5B. These figures show views of a distal end of two exemplary delivery systems according to the present invention. A proximal end of the delivery system may employ a handle as describe in connection with FIGS. 6A and 6B, discussed further below. The elongate or shaft portion of the device may have a length 150 to 180 cm. Alternatively, it may be about 300 cm long to facilitate exchange of over the wire catheters without a “dock” extension.

Regarding FIG. 5A, it shows a distal end or shaft 100 of the subject delivery system 80. The device preferably comprises a flexible atraumatic distal tip 102 of one variety or another. The tip is typically mounted to a tapered section of corewire 104. Corewire 104 may have a number of tapered sections transitioning between different diameter sections as shown.

As illustrated, a more proximal section “P” is larger in diameter than a more distal section “D” of the wire. Such an approach offers good distal flexibility, but in a robust enough wire with good pushability (column strength) and torque transmission characteristics.

The distal reduced diameter section of the wire upon which stent 82 is mounted will typically have a length of at least about 5 to 15 cm proximal of blocker 88. The length of this region is important because it defines the portion of the device with the most space between corewire 104 and outer sleeve 106. Inner sleeve 108 occupies some of this space.

It does so in each of the designs shown in FIGS. 5A and 5B. In the approach shown in FIG. 5A, a distal end 110 of the sleeve serves an additional purpose as well—as elaborated upon below. As for the sleeve occupying space up to a point adjacent to the stent (e.g., directly adjacent the stent or about a blocker's width away), it functions to control stent deployment during delivery. By providing a system with minimal internal gaps, when in tortuous anatomy and pulling/pushing members relative to one another to remove a tubular member to release a stent, the parts remain substantially coaxially aligned. With larger gaps, misalignment occurs in which components in tension are pulled into a minimum radius configuration and components in compression are pushed into a maximum radius configuration. This miss-match of action introduces unwanted variables into a stent delivery procedure which, as noted by the assignee hereof, causes forward thrust of tip 102 when delivering a stent with a system lacking sleeve 108. Not to be bound by a single theory, but it has been surmised that build-up of the alignment miss-match followed by release of static friction upon the compressed core member when sleeve 106 begins to slide back accounts for the tip thrust. Accordingly, sleeve 106 in the system offers a direct improvement to stent placement.

Naturally, if one with skill in the art were to appreciate the problem illustrated above, that person might seek to minimize system tolerances. However, at smaller system sizes, a panoply of factors must be balanced in system design. Among these are the delicacy of the parts and the difficulty in their manufacture. A tube having uniform wall thickness is therefore desired. As such, stepped tubing is not desirable. On the other hand, thick-walled, straight-gauge tubing is not desirable because its use would take away very valuable space in the stent carrying region 110, requiring greater stent compression. Further, a thicker outer sheath may deleteriously affect delivery system flexibility or tractability through tortuous anatomy.

A two-sleeve solution addresses each of these problems in a number of ways. Straight-gauge tubing can be employed to provide an advantageous combined profile. Such an approach may offer higher precision in construction as well as reduced cost. In addition, the use of two sleeves with small gaps between them has proven advantageous for flushing the system in preparation for use. Such action may be assisted by providing—in essence—multiple capillary channels to “wick-in” fluid. Flushing (and hence filling) at least the distal end of the system with saline prior to insertion in the body avoids capillary action pulling blood into the system to hamper actuation. Hydrophilic coatings may be employed to assist in this matter as well.

As referenced above, the system in FIG. 5A uses a distal end 112 of inner sleeve 108 in coordinated use with a raised feature 88 on the guidewire as a combination stent stop, blocker or abutment interface. The raised feature comprises a solid body bonded, welded, soldered or otherwise attached to the corewire or a feature ground into the wire. Combined blocker 114 is formed with the distal end of the sleeve in place. It abuts stent 82 when sleeve 106 is withdrawn to release the stent. Then, when inner sleeve 108 is removed form “bump” 88, a relatively small feature remains.

Yet, in a small diameter delivery system (in which a tapered corewire is employed), bump 88 serves a critical function by occupying space to the stent does not slip inwardly and pass inner sleeve upon outer sleeve withdrawal. In such a system according to the present invention, sleeve 108 is typically less than about 0.002 inch thick. More often, it is between about 0.0015 and about 0.001 inch thick. Relative to the stent, sleeve 108 may be between about ¼ to about ¾ the thickness of the stent.

Feature 88 and inner sleeve distal end 112 may remain aligned by virtue of the length of sleeve 108. Alternatively, a light press interference fit, adhesive, etc may be employed to temporarily lock the members together until release is intended. The length of element 88 may be between about 1 and about 5 mm. Too short a section and sleeve 108 may be prone to slip past the feature; too long a section and it may deleteriously affect flex performance of the core member.

As noted above, an advantage of the combined sleeve/core feature blocker 114 is that it offers a relatively smaller diameter “bump” remainder on the corewire after sleeve removal. This fact, in turn, facilitates the methodology referenced in FIG. 4H′ in which after stent delivery by releasing the stent from a distal portion of the outer sleeve, each of the inner and outer sleeves have been removed. With the device utilizing the combination blocker approach, the stent abutment feature then has a low enough profile that it does not interfere with subsequent use of the core member as a fully functional guidewire. In this manner, a balloon catheter or another member advanced over the core member after removal of the other system components. Especially where the abutment/blocker member diameter is larger than about 0.002 inch over an adjacent section (0.004 inch greater than diameter), ramp section(s) 116 on the proximal side of feature 88 may be provided to offer an improved transition.

In instances where inner sleeve 108 stops short of the blocker feature 88′, since that feature will serves as a stent stop or abutment alone. Therefore, as illustrated in Detail B, “bump” 88′ is significantly larger than stop/bump 88 in Detail A for proportionately-sized systems 100/100′. Again, as referenced above, with the enlarged stop feature 88′, the stripped-down system (i.e., corewire remaining after sleeve 106/108 removal) may still be used as illustrated in FIG. 4I′.

However configured, raised feature 88/88′ may comprise a gold or platinum band connected to the corewire in order to serve a marker function. A distal marker band may also be provided in the system. Such a band (not shown) may be attached to a distal end of the sleeve 106. Still further, proximal or distal section(s) 120 of tip 102 may comprise highly radiopaque platinum material. In use, the various radiopaque markers or features may be employed in the system to 1) locate stent position and length or that of other devices/features (e.g., an embolic filter), 2) indicate device actuation and stent delivery and/or 3) locate the distal end of the delivery guide. As such, various platinum (or other radiopaque material) bands or other markers (such as tantalum plugs) may be variously incorporated into the system. Alternatively, or additionally, the overhang feature serving (at least in part) stent stop or blocker member may be made of radiopaque material. Especially where the stent employed may shorten somewhat upon deployment, it may also be desirable to align radiopaque features with the expected location (relative to the body of the delivery guide member) of the stent upon deployment.

As noted above, each of the inner and outer tubular members are preferably splitable. In fact, the inner sleeve 108 may be pre-split so long as the outer sleeve 106 is unsplit over at least a portion of its length so as to support the inner member. The tubular members may be coated with a hydrophilic coating for lubricity. Materials may be selected for use in constructing the guidewire core and tubular members as commonly in other stent delivery and in other catheter systems. Exemplary materials include Nylon, LLDPE, HDPE, PET, PEEK and PTFE. In order to provide additional strength to the outer sleeve without loss of space efficiency, a construction approach as taught in U.S. patent application Ser. No. 11/147,999, filed Jun. 7, 2005 (incorporated herein by reference in its entirety) may, however, advantageously be employed.

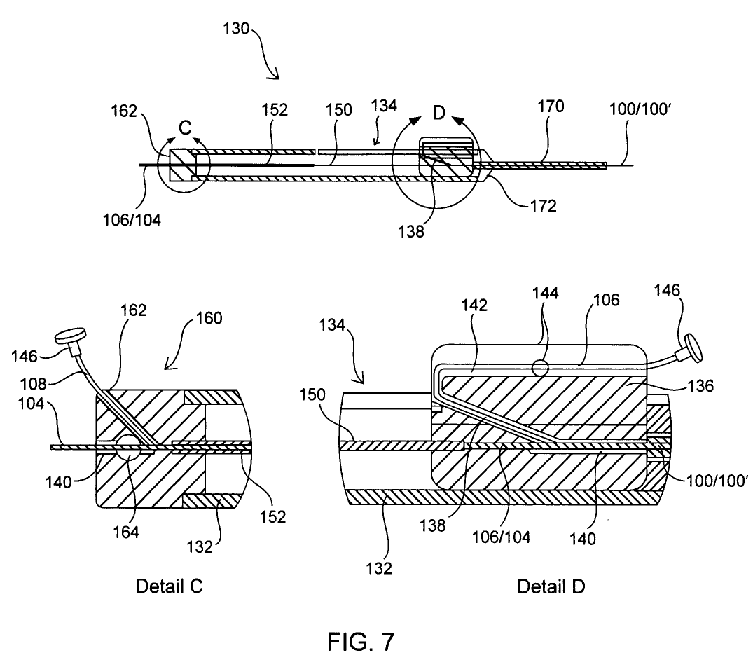

To manipulate these material layers, a handle 130 is advantageously provided as shown in FIG. 6. A cross-sectional view of the handle, together with highlighted details is shown in FIG. 7.

The handle includes a body 132 defining a slot 134 through which a slide 136 can be pulled. As shown in Detail D, the slide may includes a sleeve lumen 138 branching off of a central lumen 140 of the device through which delivery device shaft 100/100′ is received. At this point, outer sleeve 106 separates from the inner sleeve and corewire, and is received within lumen 138. Sleeve 106 is then bent over and received within channel 142 and secured to the slider via thumbscrew 144. So-configured, sleeve 106 travels with slider 136 when withdrawn through slot 134.

After stent deployment, a thumbscrew is released and sleeve 106 may be withdrawn from the assembly by grasping optional end grip and pulling. To aid in stripping sleeve 106 from the delivery system, a blade (not shown) may be incorporated to split the sleeve prior to its divergence from inner sleeve 106 and corewire 104.

Slider 136 may also receive a section of hypotube 150 received within a second piece of hypotube received by handle end plug 160. The hypotube pair 150/152 receives sleeve 106 and wire 104 providing these members under compressive force during stent deployment with support as well as protection.

Regarding plug 160, it too may include a sleeve lumen 162. In which case, inner sleeve 108 may separate from corewire 104 and be received within lumen 162. The corewire is secured to handle 130 within lumen 140 by thumbscrew/setscrew 164. Again, a grip 146 is provided to aid in removal of the sleeve upon stent deployment.

Alternatively, both of the inner sleeve 108 and corewire 104 may exit the handle in a co-axial arrangement. In which case, sleeve 106 is stripped from corewire 104 after screw 164 is released. In any case, one screw 164 is released, handle 130 may be removed from the sleeve and/or corewire. With a bare wire and no handle, the corewire wire is usable as or at least somewhat like a guidewire for a subsequent medical procedure as reference above or otherwise.

Other optional details of handle 130 may include strain relief tubing 170. These may comprise one or more tubes to ease the transition from an end cap 172 of the handle.

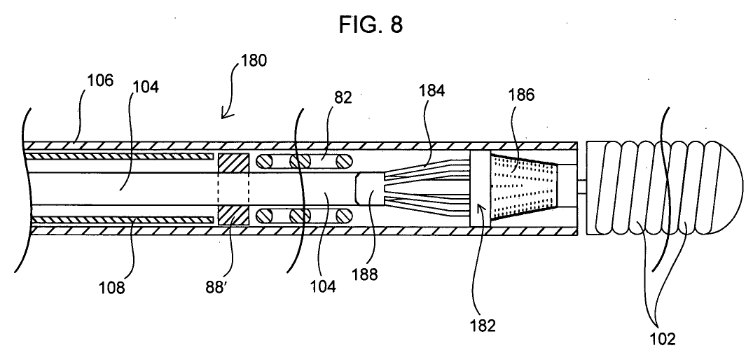

Turning now to FIG. 8, another stent delivery system 180 of the present invention is provided. System 180 includes a stent 82 and stop feature 88′ arrangement similar to that of delivery system 100′ of FIG. 5B. Naturally, the arrangement may alternatively be practiced with a stop feature arrangement as shown in FIG. 5A.

Irregardless, the system includes an optional filter component 182 on distal section 104a of corewire 104 proximal to coil tip 102. As such the system may function as a combination embolic filter and stent delivery system. Stated otherwise, a stent delivery system with embolic protection capability is provided.

In the illustrated embodiment, filter component 182 comprises an expansion frame having a plurality of outwardly biased struts 184 extending between a mesh filter 186 at a distal end and a frame base 188 coupled to corewire 104. Filter component 182 may have any suitable construct, many of which are known in the art, such as those disclosed in U.S. Pat. No. 6,027,520, incorporated herein by reference in its entirety. As such, filter component 182 may be self-expanding (as illustrated) and retained in a constrained condition by outer sheath 106 in a manner similar to the manner by which self-expanding stent 82 is constrained prior to deployment. Alternatively, filter 182 may have an active configuration driven by shape memory alloy effect. The distance between the stent and filter may vary. For a distal coronary application, however, the distance is typically between about 0.5 mm and about 5.0 mm.

In the context of the angioplasty and stent deployment procedure described with respect to FIGS. 4E-4J (and FIGS. 4D′-4I′), the use of deployment system 180 is described as follows. With as the system serving in the capacity of delivery guide 80 in FIGS. 4E-4J, distal tip 102 and filter 182 are advanced distal of the lesion 62 and beyond the distal end of outer sheath 106. Either by self-expansion, passive expansion (i.e., by blood flow within the artery) or active expansion, filter 186 is expanded (not illustrated) to operatively filter any emboli that may be released in the course of the predilatation procedure while allowing the filter blood to pass distally. The filter, then, remains deployed throughout the stent deployment and/or postdilatation procedures to capture any dislodged particulates. Stent 82 is deployed from deployment system 180 in the same manner as described above. The filter is retrieved, typically by advancing sheath 106 or the guide or balloon catheter used over the proximal portion of the device to once-again compress its shape.

Methods

The methods herein may be performed using the subject devices or by other means. The methods may all comprise the act of providing a suitable device. Such provision may be performed by the end user. In other words, “providing” (e.g., a delivery system) merely requires the end user to obtain, access, approach, position, set-up, activate, power-up or otherwise act to provide the requisite device in the subject method. Methods recited herein may be carried out in any order of the recited events which is logically possible, as well as in the recited order of events.

Variations

Exemplary aspects of the invention, together with details regarding material selection and manufacture have been set forth above. As for other details of the present invention, these may be appreciated in connection with the above-referenced patents and publication as well as generally know or appreciated by those with skill in the art.

The same may hold true with respect to method-based aspects of the invention in terms of additional acts as commonly or logically employed. In addition, though the invention has been described in reference to several examples, optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. In addition, where a range of values is provided, it is understood that every intervening value, between the upper and lower limit of that range and any other stated or intervening value in that stated range is encompassed within the invention.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in the appended claims, the singular forms “a,” “an,” “said,” and “the” include plural referents unless the specifically stated otherwise. In other words, use of the articles allow for “at least one” of the subject item in the description above as well as the claims below. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in the claims shall allow for the inclusion of any additional element—irrespective of whether a given number of elements are enumerated in the claim, or the addition of a feature could be regarded as transforming the nature of an element set forth n the claims. Stated otherwise, except as specifically defined herein, all technical and scientific terms used herein are to be given as broad a commonly understood meaning as possible while maintaining claim validity.

### CLAIMS

The breadth of the present invention is not to be limited by the examples provided and/or the subject specification, but rather only by the plain meaning of the claim terms employed. That being said,

We claim:

1. A stenting system comprising:
a self-expanding stent;
a corewire including a distal stent-receiving region, a solid raised feature on the corewire sized to abut at least a portion of the stent, and an inner sleeve and an outer sleeve set over the corewire;
wherein the corewire has a reduced distal diameter section;
wherein the inner sleeve has a proximal end located along the reduced diameter section; and
wherein the outer sleeve is adapted to restrain the stent in a compressed state.

2. The system of claim 1, wherein the distal end of the inner sleeve abuts the stent together with the raised feature.

3. The system of claim 1, wherein the distal end of the inner sleeve is proximal to the raised feature, and the raised feature is sized to abut the stent for delivery alone.

4. The system of claim 1, wherein the proximal diameter of the corewire is between about 0.009 and about 0.018 inches.

5. The system of claim 1, wherein a maximum outer diameter of the system is between about 0.018 and about 0.026 inches.

6. The system of claim 1, wherein the first and second sleeves are splittable.

7. The system of claim 6, wherein first and second sleeves are splittable up to at least about 10 cm of a distal end of each member.

8. The system of claim 6, wherein the first and second sleeves spittable along a full length of each member.

9. The system of claim 6, wherein at least a distal end of the inner sleeve is not splittable.

10. The system of claim 1, wherein the inner sleeve is pre-split.

11. The system of claim 10, wherein at least a distal end of the inner sleeve is not pre-split.

12. The system of claim 1, further comprising a filter operatively attached to the corewire at a location distal to the stent-receiving section.

13. The system of claim 11, wherein the filter comprises superelastic shape memory alloy material.

14. A stenting system comprising:
a self-expanding stent; and
a corewire including a distal stent-receiving region bordered by a proximal raised feature sized to abut at least a portion of the stent, and an inner sleeve and an outer sleeve set over the corewire;
wherein the inner and outer sleeves can be opened.

15. The system of claim 14, wherein at least one of the inner and outer sleeves are splittable.

16. The system of claim 15, wherein the inner member is pre-split.

17. A self-expanding stent delivery system comprising:
a corewire comprising a constant diameter distal section upon which a stent is received and a solid raised feature on the corewire sized to abut at least a portion of a received stent,
a filter mechanism attached to the distal section of the corewire; and
an outer sleeve set over the corewire wherein the outer sleeve is adapted to hold a stent and a filter in reduced states.

18. The system of claim 17, wherein the stent and the filter mechanism are self-expanding.

19. The system of claim 17, wherein the filter mechanism is positioned distally of the stent.

20. A method of stenting comprising:
positioning a system according to claim 1 or 14 relative to a treatment site;
delivering a stent with the system;
removing the inner and outer sleeves from the corewire; and
performing a subsequent medical procedure act with the corewire.

21. The method of claim 20, wherein the subsequent medical procedure act comprises advancing a balloon catheter over the corewire.

22. The method of claim 20, wherein the subsequent medical procedure act comprises advancing a balloon catheter over the corewire to locate a balloon across the treatment site.

23. The method of claim 22, wherein the advancing of the balloon catheter is accomplished without advancing the corewire.

24. The method of claim 22, wherein the advancing of the balloon catheter to the treatment site requires advancing the corewire.

25. The method of claim 20, wherein the removing act comprises splitting at least one of the sleeves.

26. The method of claim 20, further comprising delivering a filter with the system.

27. The method of claim 26, wherein removing the outer sleeve deploys the stent.

28. The method of claim 27, wherein the filter is deployed prior to deployment of the stent.

29. The method of claim 27, wherein the filter is deployed concurrently with deployment of the stent.

30. The method of claim 27, wherein the filter is deployed subsequent to deployment of the stent.
